(19) United States
(12) Patent Application Publication
(10) Pub. No.: US 2026/0033840 A1
(43) Pub. Date: Feb. 5, 2026

(54) MEDICAL CLIP

(71) Applicant: Aesculap AG, Tuttlingen (DE)

(72) Inventors: Thomas Scholten, Tuttlingen (DE); Jens Beger, Tuttlingen (DE); Alexander Happle, Huefingen (DE); Teresa Buening, Tuttlingen (DE); Natalia Dalmann, Tuttlingen (DE)

(21) Appl. No.: 19/356,259

(22) Filed: Oct. 13, 2025

Related U.S. Application Data
(63) Continuation of application No. PCT/EP2024/060149, filed on Apr. 15, 2024.

(30) Foreign Application Priority Data
Apr. 14, 2023 (EP) 23168050.5

(51) Int. Cl. A61B 17/122; A61B 90/00

(52) U.S. Cl. CPC A61B 17/1227; A61B 90/03; A61B 2090/034

(57) ABSTRACT

A medical clip has a first arm, second arm, and biasing element. The first arm has a first arm end connected to a first end of the biasing element by a first connecting portion. The second arm has a second arm end connected to a second end of the biasing element by a second connecting portion. The clip also has a box lock that includes the cooperating first and second connecting portions. The box lock has a first box lock perforation arranged or formed on the first connecting portion and delimited by two first box lock webs. A second box lock web of the second connecting portion passes through the first box lock perforation. The first box lock perforation has two female bearing surfaces facing toward one another. The second box lock web has two male bearing surfaces facing away from one another and toward the female bearing surfaces.

### CROSS-REFERENCE TO RELATED APPLICATIONS

This application is a continuation of International Application No. PCT/EP2024/060149, filed on Apr. 15, 2024, and claims priority to European Application No. 23168050.5, filed on Apr. 14, 2023. The contents of International Application No. PCT/EP2024/060149 and European Application No. 23168050.5 are incorporated by reference herein in their entireties.

### FIELD

The present disclosure relates to a medical clip, in particular in the form of an aneurysm clip, which comprises a first clamping arm, a second clamping arm, and a biasing element having a first and a second end, wherein the first clamping arm has a first clamping arm end, which is connected to the first end of the biasing element by way of a first connecting portion, wherein the second clamping arm has a second clamping arm end, which is connected to the second end of the biasing element by way of a second connecting portion, wherein the clip comprises a box lock comprising the cooperating first and second connecting portions, said box lock having at least one first box lock perforation arranged or formed on the first connecting portion and delimited by two first box lock webs and having at least one second box lock web comprised by the second connecting portion and passing through the first box lock perforation, wherein the first box lock perforation comprises two female bearing surfaces facing toward one another, and wherein the at least one second box lock web comprises two male bearing surfaces facing away from one another and toward the female bearing surfaces, wherein at least two bearing surface pairs are each formed having a female and a male bearing surface, and wherein in each case the cooperating female and male bearing surfaces extend in parallel with one another and define a bearing plane.

### BACKGROUND

A medical clip of the kind described at the outset is known, e.g., from WO 2022/096357 A1. A further medical clip having a box lock is described in DE 20 2004 015 274 U1. In this clip, the biasing element also takes on the function of a bearing pin for the two cooperating joint rings, which are rotatable about a longitudinal axis of the biasing element and on each of which a respective clamping arm is arranged or formed. The joint ring 23 together with the guide plate hereby forms the box lock perforation, and the joint ring 22 forms the second connecting portion passing through the box lock perforation. Furthermore, DE 20 2004 015 274 U1 discloses very similar medical clips having a box lock depicted in FIGS. 1 and 2 and described in detail in DE 199 35 418 A1.

In a connection region of aneurysm clips, in particular when the connection regions are configured in the form of box locks, the connection components move past one another when the clips are opened and closed, i.e., the box lock webs on the first connecting portion and the at least one second box lock web on the second connecting portion. In particular, they slide on one another. This movement causes friction. In other words, the box lock webs slide with their male and female cooperating bearing surfaces over one another when opening and closing the clip, which leads to friction, but also to abrasion and to cold welding, so-called seizing. These effects lead to increased repeat inaccuracy in the measurement of the closing force of the clips. Or in other words, it leads to a reduced repeat accuracy in the measurement of the closing force.

For such clips, it is important that the biasing force exerted by the biasing element, thus in particular a spring force, has a predefined value. To determine the spring force of aneurysm clips, they are opened to a certain value in accordance with an existing standard and placed on a test device. The force determined in this process, namely the above-mentioned closing force, must then be within a certain tolerance window specified by the standard. Otherwise, the clip must be revised or discarded as unusable.

The closing force of the clip must be kept within normative limits, as stated. A maximum deviation must not be more than +/−7.5% of a rated value. In addition, the closing force must not decrease even by a small amount after repeated opening. A predetermined closing force loss must be less than 5%. The measuring principle for determining the closing force and the specified limits or deviations are described in the standard EN ISO 9713:2022.

The fundamentally unavoidable friction and the associated repeat inaccuracy in the production of clips leads to the loss of an important part of the availably tolerance in manufacturing. However, this tolerance is desperately needed as a process window in production.

It is known that the harder the cooperating male and female bearing surfaces of a bearing surface pair are pressed against one another, the more friction is created. This pressure cannot be reduced at will, however, because the cooperating clamping arms, also referred to as jaw parts, then obtain undefined play relative to one another.

### SUMMARY

It is therefore an object of the present disclosure to improve a medical clip of the kind described at the outset such that a repeat inaccuracy in the measurement of the closing force is reduced.

This object is achieved, in accordance with the present disclosure, in a medical clip of the kind described at the outset in that a first box lock web projection area defined by a perpendicular projection of the first box lock webs into the bearing plane and a second box lock web projection area defined by a perpendicular projection of the at least one second box lock web into the bearing plane overlap and define a projection overlap area, in that at least one of the two cooperating bearing surfaces of at least one, in particular of each, of the at least two bearing surface pairs has at least one recess such that an actual size of the bearing surface parallel to the bearing plane, said bearing surface being defined by the box lock web comprising the at least one recess, is smaller than the box lock web projection area projected from the box lock web comprising the at least one recess into the bearing plane, in that the two cooperating bearing surfaces of at least one, in particular of each, bearing surface pair that are able to abut against one another or that are abutting in surface-to-surface contact against one another define a contact overlap area, and in that a ratio of the contact overlap area and the projection overlap area is in a range of about 1/25 to about 1/3.

The proposed further development enables, in particular, a reduction of the friction that occurs between cooperating bearing surfaces of the at least two bearing surface pairs. The bearing surfaces sliding on one another in a planar manner, which are reduced relative to the dimensions of the respective box lock webs, lead to a reduction of the static friction and facilitate the transition to sliding friction. To measure the closing force, for example, the clip can be opened slightly so that the clamping arms can be placed on two retaining pins. To measure the closing force, the clip is closed again, whereby the sliding process in the connection region changes to a static measuring state with static friction. The lower the coefficient of friction during the transition from sliding friction to static friction, the lower the friction torque during the closing force measurement and thus the influence on the measurement of the closing force and thus the dispersion of the measurement results. By reducing the contact overlap area compared to the projection overlap area in the specified region, friction can be minimized, and thus a repeat inaccuracy can be reduced as desired. In order to achieve the reduction of the contact overlap area compared to the projection overlap area, either one of the two cooperating bearing surfaces must be reduced to a value in a range of about 1/25 to 1/3 relative to the associated box lock web projection area. Alternatively, the two cooperating bearing surfaces can each be reduced in size to between about 1/5 and about 0.58. This reduction can be achieved by the at least one recess provided in one of the two bearing surfaces or optionally in both bearing surfaces. Preferably, the ratio of the contact overlap area and the projection overlap area is in a range of about 10% to about 20%. In the specified ratio ranges, a sufficiently good guidance can still be achieved by placing the bearing surfaces against one another, but with reduced friction as desired. In particular, the ratio of the contact overlap area and the projection overlap area may be in a range of about 1/25 to about 1/4 to further reduce the undesirable friction effects.

The friction can be reduced in a simple manner if the at least one recess is configured in the form of a groove in the bearing surface or in the form of a chamfer on the bearing surface. In particular, the bearing surface may be formed along two side edges extending in parallel or substantially in parallel, said side edges extending, in particular, in parallel with an extent of the respective connecting portion from the biasing element to the respective clamping arm. Thus, one or two chamfers can be provided on the respective box lock web to reduce an actual size of the bearing surface in cooperation with the bearing surface associated therewith, which forms a bearing surface pair. The chamfer may, in particular, define a planar surface or define a curved surface, which may be convexly or concavely curved pointing away from the respective connecting portion.

Preferably, the groove or the chamfer extends in parallel or substantially in parallel with a longitudinal direction defined by the respective box lock web. For example, the groove may pass through longitudinal edges of the bearing surface if the groove extends in an S-shape from the biasing element to the clamping arms.

It is favorable if the chamfer defines an inclined surface that together with the bearing plane encloses an obtuse chamfer angle. In particular, the inclined surface may extend in a planar or curved manner. The chamfer angle, in particular, may have a value in a range of about 140° to about 175°. As proposed, this results, for example, in an almost straight angle in the transition of the bearing surface to the chamfer, which avoids burrs. It is favorable if the chamfer angle is in a range of about 165° to about 172°.

Further, it may be advantageous if the groove extends transversely, in particular perpendicularly, to a longitudinal direction defined by the respective box lock web. In particular, a plurality of grooves can thus be provided. In particular, these can extend in parallel with one another. Furthermore, the grooves may have a circular segment shape in cross section, such that, in particular, an obtuse, preferably straight angle is created in the transition to the remaining bearing surface. This prevents burrs during the production of the clip.

It is further favorable if the at least one recess is configured in the form of a hollow sphere portion. In particular, the hollow sphere portion, also referred to as a hollow sphere segment, has the form of a half hollow sphere or a portion of a half hollow sphere, such that a recess is formed free of undercuts. Such recesses can be formed in a simple manner, for example with a ball cutter. Preferably, a depth of the hollow sphere portion is significantly smaller than a radius thereof. In particular, the depth is less than 1/5 of the radius.

It is further favorable if the at least one recess is formed by a box lock web perforation passing through the box lock web. For example, such a box lock web perforation may be formed by a bore passing through the box lock web.

The box lock web perforation can be formed in a simple manner if it has a circular cross section. In particular, it can thus be formed by a bore.

In order to reduce the actual effective bearing surface in comparison to the respective box lock web projection area in the desired manner, it is advantageous if at least one of the two cooperating bearing surfaces of at least one, in particular of each, of the at least two bearing surface pairs has a plurality of recesses. For example, the size of the bearing surface that actually contributes to the friction of the respective bearing surface pair can thus be reduced as desired, and a risk of the cooperating bearing surfaces jamming can be minimized.

The medical clip can be formed in a simple manner if the plurality of recesses are of identical configuration.

In order to optimize the reduction of the bearing surface, it is favorable if at least a portion of the plurality of recesses differ. In particular, a difference with respect to the shape and/or size of the recesses is conceivable. In particular, both hollow spherical recesses and grooves may be provided on the same bearing surface. For example, more than two grooves of different shape and size may be provided and also more than two recesses configured in the form of a hollow sphere portion.

In order to achieve the smallest possible contact overlap area, it is advantageous if both cooperating bearing surfaces of each bearing surface pair comprise at least one recess.

In accordance with a further preferred embodiment of the present disclosure, provision may be made that a width of the box lock webs, without taking into account the at least one recess, transverse to a longitudinal direction defined by the respective box lock web is in a range of about 0.9 mm to about 2.1 mm.

Further, it is advantageous if the first clamping arm and the second clamping arm in a basic position of the clip are maximally proximate to one another, in particular abutting against one another, and are movable away from one another against the action of the biasing element from the basic position into an opening position. The biasing element then exerts a force on the two clamping arms in order to automatically transfer them from the opening position back into the basic position when the clamping arms are released again by a user after opening. Exactly this force is then the closing force that is to be predetermined for the clip and measured with the highest possible repeat accuracy or, in other words, with the lowest possible repeat inaccuracy.

An optimal function of the medical clip can be achieved, in particular, when the bearing plane is of planar or substantially planar configuration. Thus, the cooperating bearing surfaces are also of planar or substantially planar configuration.

In accordance with a preferred embodiment of the present disclosure, provision may be made that the box lock is configured in the form of a single box lock and that the second connecting portion comprises only one second box lock web. The single second box lock web can then pass through the first box lock perforation and is hereby laterally delimited by the two first box lock webs with their female bearing faces facing toward one another.

Further, it is favorable if the box lock is configured in the form of a double box lock, if the second connecting portion comprises two second box lock webs, which laterally delimit a second box lock perforation, if one of the two first box lock webs passes through the second box lock perforation, and if one of the two second box lock webs passes through the first box lock perforation. Double box locks have the advantage, in particular in the case of medical clips with particularly long clamping arms or jaw parts, that they help to largely or completely avoid undesired “scissoring”, i.e., the sliding of lateral edges of the clamping arms on one another when closing the clip. “Scissoring” carries the risk that soft tissue can be injured and, in the worst case, severed in an undesirable way. This should be avoided wherever possible.

Preferably, the first clamping arm commencing from the first clamping arm end and the second clamping arm commencing from the second clamping arm end are of rectilinear or curved or angled configuration in the direction toward free ends thereof. Medical clips can thus be formed having jaw parts of almost any shape in order to optimally treat aneurysms of various shapes and sizes in human or animal patients.

The clip is preferably made of a metallic material. In particular, a stability of the clip can thus be ensured as necessary. It is favorable if the metallic material is titanium or an alloy containing titanium, for example Ti6Al4V. Thus, it is advantageous if the clip is made of a biocompatible material in order to avoid rejection reactions.

In particular if the bearing surfaces are made of titanium or an alloy containing titanium, a large contact overlap area between the two cooperating bearing surfaces leads to undesirably high friction. Therefore, as described, a reduction of at least one bearing surface of one of the at least two bearing surface pairs in comparison to the projected box lock web projection area is advantageous.

Preferably, the cooperating bearing surfaces are made of a metallic material. In particular, it may be titanium or an alloy containing titanium. It is therefore advantageous if the cooperating bearing surfaces are made of a biocompatible material.

In accordance with a further preferred embodiment of the present disclosure, provision may be made that the biasing element is configured in the form of a coil spring with at least one winding. In particular, the coil spring may be configured having at least about 1.5 windings. For example, the biasing element can be produced from a blank formed by press forming by winding.

The production of the medical clip can be further simplified, in particular the formation of planar bearing surfaces, if the first and/or second box lock webs, without considering the at least one recess, have a rectangular cross section.

Furthermore, it may be advantageous if at least one bearing surface, in particular only one bearing surface or both bearing surfaces, of the two cooperating bearing surfaces of a bearing surface pair is provided with a coating. In particular, the coating may be configured in the form of a corrosion-reducing coating or a passivating coating.

In particular, it is favorable if the coating is configured to reduce friction. Thus, in addition to the proposed reduction of the size of the bearing surface by providing at least one recess, the undesirable friction that occurs between cooperating bearing surfaces of a bearing surface pair can be further reduced by the friction-reducing coating.

The coating is preferably configured in the form of an oxide layer. For example, it may be titanium oxide if the medical clip is made of a material containing titanium. Such an oxide layer is, in particular in the case of titanium, much harder than the base material of the clip and therefore much less prone to seizure when the cooperating bearing surfaces slide against one another.

The oxide layer can be applied galvanically in a simple manner.

The medical clip is preferably configured in the form of an aneurysm clip. In particular, it can thus be used to treat aneurysms, in particular sacculations on hollow organs.

### BRIEF DESCRIPTION OF THE DRAWINGS

The subsequent description of preferred embodiments of the present disclosure serves in conjunction with the drawings for further explanation.

FIG. 1 shows a schematic perspective total view of a first embodiment of a medical clip having a single box lock;

FIG. 2 shows a schematic perspective total view of a second embodiment of a medical clip having a double box lock;

FIG. 3 shows a schematic partial view of a first connecting portion of the medical clip depicted in FIG. 1;

FIG. 4 shows a schematic partial view of a second connecting portion of the medical clip from FIG. 1;

FIG. 5 shows schematic section view of the medical clip from FIG. 1 in the region of the box lock;

FIG. 6 shows a schematic section view of the medical clip from FIG. 2 in the region of the box lock;

FIG. 7 shows a schematic section view similar to FIG. 5 of a further embodiment of a medical clip;

FIG. 8 shows a schematic section view similar to FIG. 6 of a further embodiment of a medical clip;

FIG. 9 shows a schematic partial view similar to FIG. 3 of first connecting portion of a further embodiment of a medical clip;

FIG. 10 shows a section view along line 10-10 in FIG. 9;

FIG. 11 shows a schematic partial view similar to FIG. 4 of a second connecting portion of a further embodiment of a medical clip;

FIG. 12 shows a schematic, partially broken plan view of the box lock region of an embodiment of a medical clip, which is formed by the first and second connecting portions depicted in FIGS. 9 and 11;

FIG. 12A shows an enlarged schematic view of the region A in FIG. 12 for illustrating overlapping box lock web projection areas, which form the projection overlap area, and for illustrating the contact overlap area of two cooperating bearing surfaces;

FIG. 13 shows a schematic partial view similar to FIG. 9 of first connecting portion of a further embodiment of a medical clip;

FIG. 14 shows a section view along line 14-14 in FIG. 13;

FIG. 15 shows a schematic partial view similar to FIG. 11 of a second connecting portion of a further embodiment of a medical clip;

FIG. 16 shows a schematic partial view similar to FIG. 9 of first connecting portion of a further embodiment of a medical clip;

FIG. 17 shows a section view along line 17-17 in FIG. 16;

FIG. 18 shows a schematic partial view similar to FIG. 11 of a second connecting portion of a further embodiment of a medical clip; and

FIG. 19 shows a schematic section view similar to FIG. 5 of a further embodiment of a medical clip.

### DETAILED DESCRIPTION

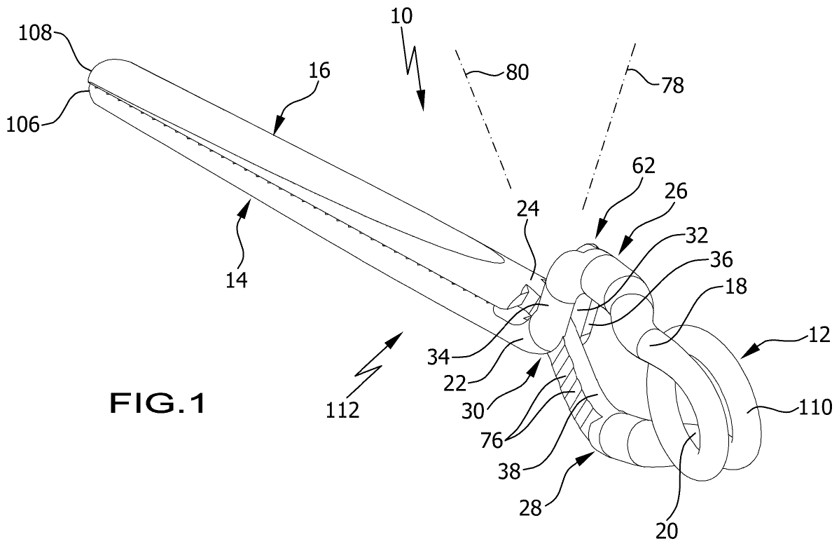

A first embodiment of a medical clip is schematically depicted in FIG. 1 and is denoted as a whole with the reference numeral 10.

The medical clip 10 comprises a biasing element 12 and two clamping arms, namely a first clamping arm 14 and a second clamping arm 16.

The biasing element 12 has a first end 18 and a second end 20. The first clamping arm 14 has a first clamping arm end 22. The second clamping arm 16 has a second clamping arm end 24.

The first end 18 of the biasing element 12 is connected to the first clamping arm end 22 by way of a first connecting portion 26. The second end 20 of the biasing element 12 is connected to the second clamping arm end 24 by way of a second connecting portion 28.

A connection region of the clip 10 is configured in the form of a box lock 30, which comprises the two connecting portions 26 and 28.

The first connecting portion 26 defines a box lock perforation 32, which is laterally delimited by two first box lock webs 34 and 36. The second connecting section 28 comprises one single second box lock web 38 passing through the first box lock perforation 32.

The first box lock perforation 32 comprises two female bearing surfaces 42 and 44 that face toward one another. The second box lock web 38 comprises two male bearing surfaces 46 and 48 that face away from one another and toward the female bearing surfaces 42 and 44.

The box lock 30 comprises two bearing surface pairs 50 and 52. The bearing surface pair 50 comprises the female bearing surface 42 and the male bearing surface 46. The bearing surface pair 52 comprises the female bearing surface 44 and the male bearing surface 48.

The cooperating female and male bearing surfaces 42, 46 and 44, 48 respectively in each case extend in parallel with one another and in each case define a respective bearing plane 56 and 58. In this embodiment, the bearing planes 56 and 58 also extend in parallel with one another.

The box lock 30 is configured in the form of a single box lock 62. The second connecting portion 28 comprises only one single box lock web, namely the second box lock web 38.

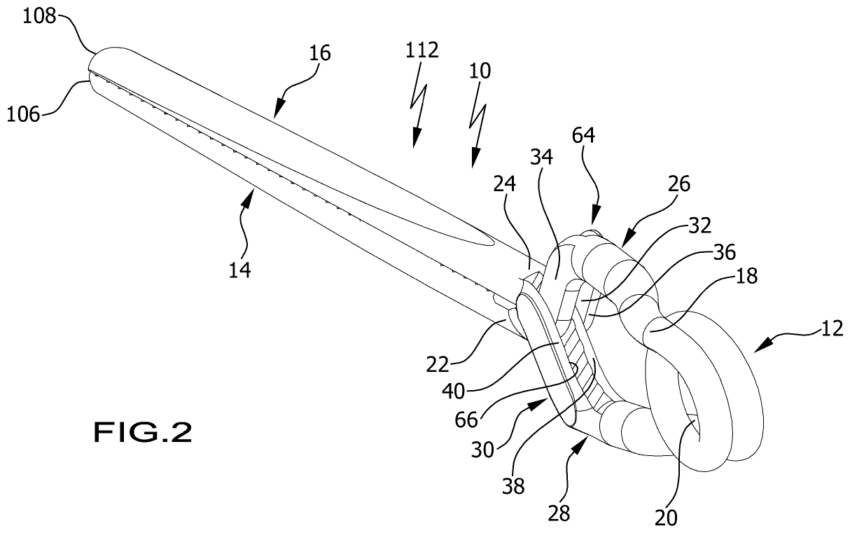

A second embodiment of a medical clip 10 is schematically depicted in FIG. 2. This clip 10 substantially corresponds in its structure to the embodiment of the clip 10 from FIG. 1, such that in the embodiment of FIG. 2 the same reference numerals are used for designating identical or functionally comparable components and elements.

The medical clip 10 from FIG. 2 differs from the first embodiment of the medical clip from FIG. 1 in the design of the box lock 30. In the embodiment of FIG. 2, the box lock 30 is configured in the form of a double box lock 64. The second connecting portion 28 in this case comprises two second box lock webs 38 and 40. The second connecting portion 28 further comprises a second box lock perforation 66. Said perforation is delimited laterally by the two second box lock webs 38 and 40. In the case of this double box lock 64, the box lock web 38 passes through the first box lock perforation 32 in the same way as in case of the clip 10 in FIG. 1. However, in the case of the double box lock 64, the first box lock web 34 passes through the second box lock perforation 66.

As schematically illustrated in FIG. 6, a further female bearing surface 68 is formed on the second box lock web 40, which cooperates with a male bearing surface 70 on the first box lock web 34. In the case of this double box lock 64, the male bearing surface 48 thus fundamentally forms a female bearing surface of the second box lock perforation 66. In this case, in an analogous manner, the female bearing surface 44 forms in its function a male bearing surface of the first box lock web 34 in cooperation with the bearing surface 48.

The double box lock 64 comprises three bearing surface pairs 50, 52, and 54. The bearing surface pair 50 is formed by the cooperating bearing surfaces 42 and 46. The bearing surface pair 52 is formed by the cooperating bearing surfaces 44 and 48. The bearing surface pair 54 is formed by the cooperating bearing surfaces 68 and 70.

The problem of known medical clips described at the outset, namely that they have insufficient repeat accuracy in determining the closing force, is solved in embodiments of medical clips 10 described the following in connection with the Figures in that at least one of the cooperating bearing surfaces 42, 46, 44, 48 and optionally 68, 70 has at least one recess to reduce the effective size of the bearing surface provided with at least one recess 72 in cooperation with the corresponding associated bearing surface, such that the size of a contact overlap area 74 is also reduced. The minimum requirement is that at least one of the bearing pairs 50, 52 and optionally 54 has at least one bearing surface with at least one recess in the case of a clip 10 with a double box lock 64. The contact overlap area 74 is defined by the surface region in which the two cooperating bearing surfaces 42 and 46 or 44 and 48 or 68 and 70 of the bearing surface pairs 50, 52, or 54 that are able to abut against one another or that are abutting in surface-to-surface contact against one another are in contact with one another or slide on one another when opening and closing the clip 10.

The at least one recess 72 may be configured in a different form, as is described in the following in connection with the Figures.

As defined, the contact overlap area 74 lies in the respective bearing plane 56, 58 and 60. The bearing plane 60 is defined by the cooperating bearing surfaces 68 and 70.

It should be noted beforehand that the box lock web 38 in the case of the single box lock 62 and the box lock webs 34 and 38 in the case of the double box lock 64 have a rectangular cross-section without taking into account the at least one recess 72. A cross section of the box lock webs 34 and 36 in the clips 10 with a single box lock 62 is substantially elongated oval. The cross sections of the box lock webs 36 and 40 are each approximately rectangular in the case of the double box lock 64.

The embodiment of the clip 10 according to FIGS. 1, 3, 4, and 5 has a plurality of recesses 72. The recesses 72 are configured in the form of grooves 76 in the bearing surfaces 42 and 46, which in the case of the clip 10 cooperate with one another and define the bearing surface pair 50. In the embodiment of FIGS. 1, 3, 4, and 5, the grooves 76 extend in parallel with one another and transversely to longitudinal directions 78 and 80 respectively, which are defined on the one hand by the first box lock webs 34 and 36 and on the other hand by the box lock web 38.

In the case of the embodiment depicted, the grooves 76 are milled in order to thus achieve a smaller surface area, and thus a smaller effective size of the bearing surfaces 42 and 46 so as to also reduce the friction between them.

In cross section, the grooves 76 have a boundary line that forms a circular arc section. This results in an obtuse, almost stretched angle in the transition to bearing surfaces 42 and 46. The formation of burrs is avoided in this way.

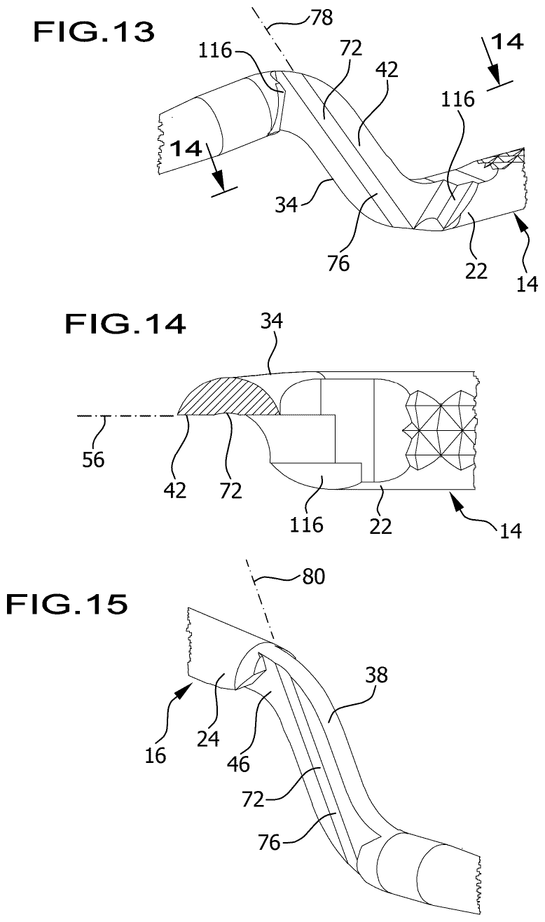

In the embodiment of a medical clip 10 that is partially schematically depicted in FIGS. 13 to 15, only one single groove 76 is formed in the bearing surfaces 42 and 44 respectively. Said groove extends in each case in parallel or substantially in parallel with the respective longitudinal directions 78 and 80 defined by the respective box lock webs 34 and 38. The grooves in this embodiment, too, are delimited in cross section by a line that defines a portion of a circular arc, such that again an obtuse, nearly straight angle is defined in the transition to the remaining bearing surface 42 or 46 that is reduced in size. The abutting bearing surfaces 42 and 46 in the common bearing plane 56 are reduced to the region of the contact overlap area 74, thereby reducing the friction created between the bearing surfaces 42 and 46 compared to bearing surfaces 42 and 46 without the groove 76.

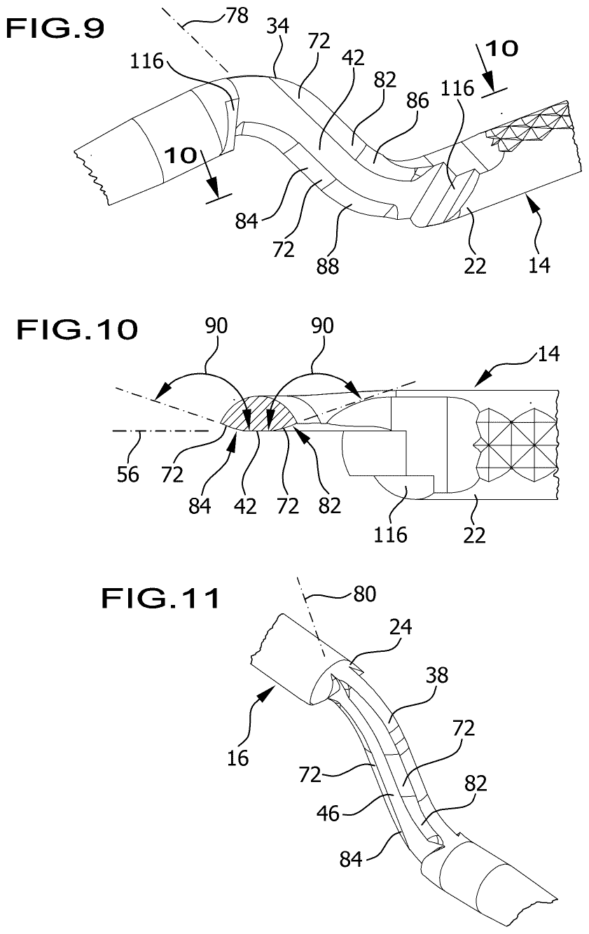

In a further embodiment, which is schematically illustrated in FIGS. 9 to 11, two respective recesses 72 in the form of chamfers 82 and 84 are formed on the box lock webs 34 and 38. These chamfers 82 and 84 are formed on the bearing surface 42 and 46 respectively and reduce an effective size for bearing on another, the size extending in parallel with the bearing plane 56. Thus, only a narrow strip of the bearing surface 42 parallel to the bearing plane 56 and a narrow strip of the bearing surface 46 parallel to the bearing plane 56 remain on the closing web 38. The chamfers 82 and 84 define inclined surfaces 86 and 88, which together with the bearing plane 56 enclose an obtuse chamfer angle 90. The chamfer angle 90 has a value in a range of about 140° to 175°. In the embodiment depicted in the Figures, the chamfer angle 90 is in a range of about 165° to 172°.

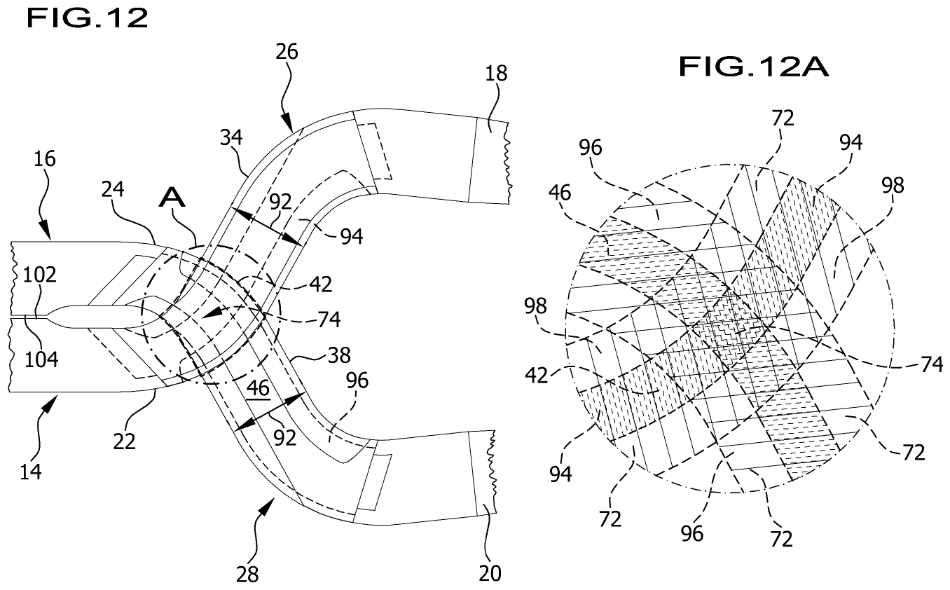

Due to the formation of chamfers 82 and 86, as described, only narrow strips of the bearing surfaces 42 and 46 remain, having a strip width that corresponds to about 1/3 of a width 92 of the box lock webs 34 and 38. The two bearing surfaces 42 and 46 can thus abut against one another in surface-to-surface contact only in a region defined by the contact overlap area 74. The contact overlap area 74 is depicted schematically in FIGS. 12 and 12A as an overlap region of the remaining bearing surfaces 42 and 46. In FIG. 12A it is marked by double dashed hatching.

A first box lock web projection area 94 is defined by a perpendicular projection of the first box lock web 34 into the bearing plane 56. A perpendicular projection of the second box lock web 38 into the bearing plane 56 defines a second box lock web projection area 96. The box lock web projection areas 94 and 96 overlap in the bearing plane 56 and thus define a projection overlap area 98. This is shown schematically in FIGS. 12 and 12A and is shown in FIG. 12A with double hatching. The contact overlap area 74, which forms a portion of the projection overlap area 98, on the other hand, is shown quadruple hatched by superimposing the double hatching of the projection overlap area 98 and the double dashed hatching.

In this embodiment, the two bearing surfaces 42 and 46 of the bearing surface pair 50 have at least one recess 72, namely in each case two recesses 72 in the form of chamfers 82 and 84, such that an actual size of the bearing surfaces 42 and 46 in parallel with the bearing plane 56 is smaller than the box lock web projection areas 94 and 96 projected from the box lock webs 34 and 38 into the bearing plane 56. A ratio of the contact overlap area 74 and the projection overlap area 98 is in a range of about 1/25 to about 1/3. In the embodiment as schematically illustrated in FIGS. 9 to 12, the ratio is about 1/9 assuming that about a third of the respective bearing surfaces 42 and 46 parallel to the bearing plane 56 is removed in each case by the two chamfers 82 and 84. The two remaining bearing surfaces 42 and 46, which compared to the original bearing surfaces 42 and 46, i.e., without taking into account the chamfers 82 and 84, thus without taking into account the two recesses 72, have the width 92, are each reduced to a third relative to the widths of the first and second box lock web projection areas 94 and 96. When the bearing surfaces 42 and 46 abut against one another, this results in the product of the remaining bearing surfaces 42 and 46, each reduced to about 1/3 of their original size, and thus a size of the contact overlap area 74 that corresponds to only about 1/9 of the size of the projection overlap area 98.

The described view of the reduction of the size of the contact overlap area 74 taking into account the formation of at least one recess 72 in one of the two cooperating bearing surfaces of at least one bearing surface pair 50, 52 or 54 is correspondingly applicable to other forms of recesses 72. Thus, the bearing surfaces 42 and 46 can also be reduced in a similar manner when the grooves 76 are formed transversely to the longitudinal directions 78 and 80, wherein then not only one single contact overlap area 74 is defined, but instead a multitude of correspondingly smaller contact overlap areas 74, which then in sum have an area value that is in a ratio in a range of about 1/25 to about 1/3 relative to the projection overlap area 98 determined as explained above. Also for grooves 76 that extend in parallel with the longitudinal directions 78 and 80, an analogous view is obtained. In this case, four contact overlap areas 74 are created by the two respective strips remaining from the bearing surfaces 42 and 46, said strips being separated from one another by the groove 76, coming into contact with one another, wherein a ratio of the area sum of the four contact overlap areas 74 is then about 1/25 to about 1/3 relative to the projection overlap area 98.

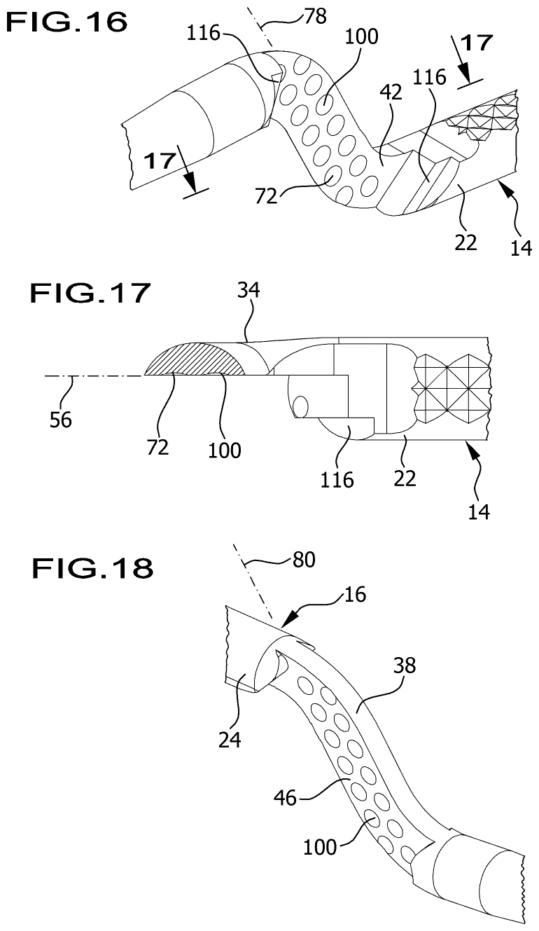

FIGS. 16 and 18 schematically show a section of the connecting portions 26 and 28 of a clip 10. In this embodiment, a plurality of recesses 72 are formed in the bearing surfaces 42 and 46, namely in the form of hollow sphere portions 100. In the illustrated embodiment, a depth of the recess is substantially smaller than a radius of the hollow sphere portions 100. In particular, the hollow sphere portions 100 may have the shape of a half hollow sphere. Here, too, the recesses 72 are milled so that an obtuse, almost straight angle is obtained in the transition to the remaining bearing surfaces 42 and 46.

Also in this embodiment, the remaining bearing surfaces 42 and 46, taking into account the recesses 72, are significantly reduced in comparison to the bearing surfaces 42 and 46 without taking into account the recesses 72. Without taking into account the recesses 72, first and second box lock projection areas 94 and 96 in the bearing plane 56 are also created here as explained above. For the abutting bearing surfaces 42 and 46 with the recesses 72 in the form of hollow sphere portions 100, taking into account the recesses 72, only a contact overlap area 74 remains in a size such that here, too, a ratio of the contact overlap area 74 and the projection overlap area 98 having a value in a range of about 1/25 to about 1/3 is obtained.

Alternatively, the at least one recess 72 may also be formed by a box lock web perforation passing through the respective box lock web 34 or 38. Such box lock web perforations result in a view of the bearing surfaces 42 and 46 analogous to FIGS. 16 and 18, namely when the box lock web perforations have a circular cross section.

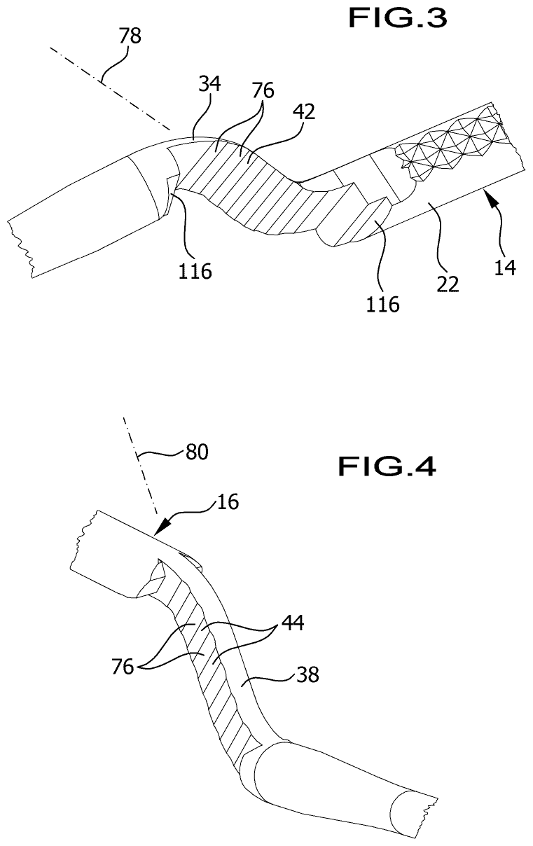

In the embodiments of FIGS. 3 and 4, 9 to 11, and 16 to 18, at least one of the two cooperating bearing surfaces 42 and 46 of the bearing surface pair 50 has a plurality of recesses 72.

In the embodiments of FIGS. 3 and 4 on the one hand and 16 to 18, the plurality of recesses 72 are each of identical configuration.

In embodiments not shown in the Figures, at least a portion of the plurality of recesses 72 is configured differently, optionally differing in their shape and/or their size.

The embodiments described so far provide that the two cooperating bearing surfaces 42 and 46 of the bearing surface pair 50 each comprise at least one recess 72.

The width 92 of the box lock webs 34 and 38, without taking into account the at least one recess 72, transverse to the longitudinal directions 78 and 80 defined by the respective longitudinal web 34 and 38, has a value that is in a range of about 0.9 mm to about 2.1 mm. For example, the clips 10 may have altogether different sizes, such that the width 92, which is ultimately predetermined by a diameter of a wire that forms the starting material for producing the clips 10, has a value which, for example, may be about 1 mm, about 1.2 mm, or about 1.7 mm.

As schematically depicted in FIGS. 1 and 2, the clamping arms 14 and 16 are maximally proximate to one another in a basic position of the respective clip 10. FIGS. 1 and 2 show embodiments of clips 10 in which the clamping arms 14 and 16 abut against one another in the basic position.

The clamping arms 14 and 16 can be moved away from one another from the basic position into an opening position against the action of the biasing element 12, so that a sacculation of a hollow organ is able to be introduced between clamping surfaces 102 and 104 of the clamping arms 14 and 16 and can be clamped therebetween, namely by releasing the clip 10 so that the biasing element 12 is able to exert a biasing force to press the clamping surfaces 102 and 104 of the clamping arms 14 and 16 against one another.

The clamping arms 14 and 16 extend from the first clamping arm end 22 and from the second clamping arm end 24 respectively in the direction toward free ends 106, 108.

In the embodiments of the clips 10 of FIGS. 1 and 2, the clamping arms 14 and 16 extend rectilinearly. In alternative embodiments that are not depicted, the clamping arms 14 and 16 are curved or angled, wherein in each case it must be ensured that the clamping surfaces 102 and 104 in the basic position abut against one another in surface-to-surface contact substantially over the entire extent from the clamping arm ends 22 and 24 to the free ends 106 and 108.

In the embodiments depicted in FIGS. 1 and 2, the biasing element 12 is configured in the form of a coil spring 110 with at least one winding. The embodiments of FIGS. 1 and 2 comprise coil springs 110 with about 1.5 windings.

In all embodiments, the medical clip 10 is configured in the form of an aneurysm clip 112.

The clip 10 is made of a metallic material. In the embodiments depicted in the Figures, the clip 10 is made of titanium or an alloy containing titanium, for example Ti6Al4V.

A clip 10 made from said metallic materials has bearing surfaces 42, 44, 46 and 48 and optionally 68 and 70, which are then made from the metallic material, namely titanium or an alloy containing titanium.

At least one of the bearing surfaces 42, 44, 46 and 48 and optionally 68 and 70, which form the bearing surface pairs 50, 52 and optionally 54, is provided with a coating in a manner not described in more detail. In particular, in each case at least one bearing surface of each bearing surface pair 50, 52 and optionally 54 is provided with a coating. Such a coating is an optional configuration of the clips 10. For example, only one of the bearing surfaces 42 and 46 or both bearing surfaces 42 and 46 may be provided with a coating. This also applies accordingly to further bearing surface pairs 52 and optionally 54.

The coating is preferably configured to reduce friction.

In the case of metallic materials used for the production of the clip 10, the coating is preferably configured in the form of an oxide layer. In the case of clips 10 made of titanium or an alloy containing titanium, the oxide layer is preferably a titanium oxide layer.

The oxide layer for coating the bearing surfaces 42, 44, 46 and 48 and optionally 68 and 70 is preferably applied galvanically.

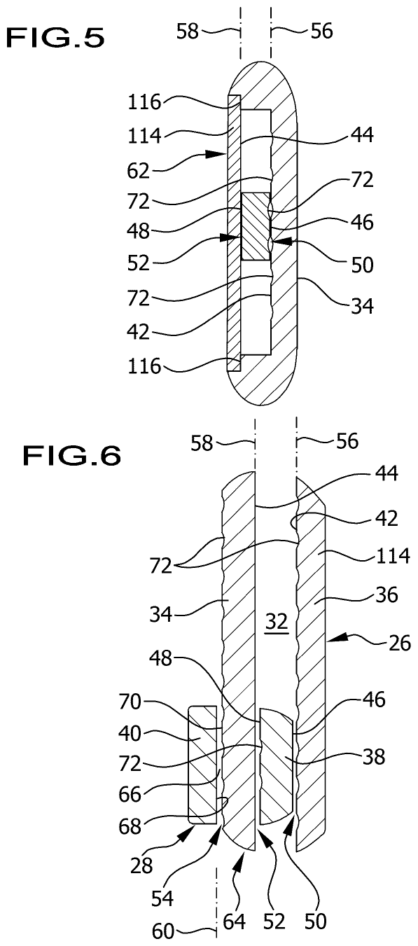

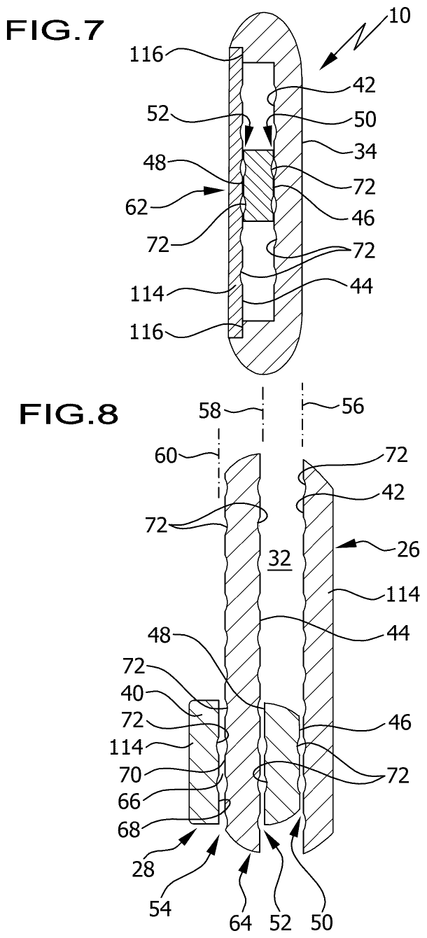

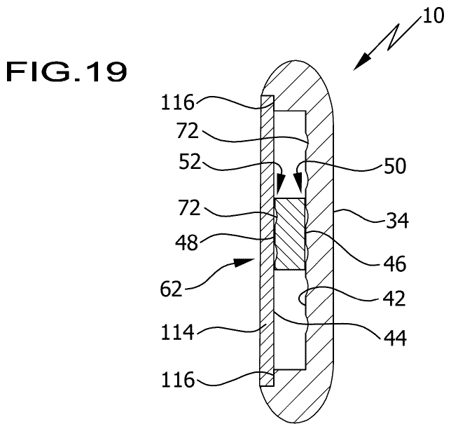

FIGS. 7 and 19 schematically show further embodiments of a clip 10 with single box lock 62. In the embodiment of FIG. 7, as in the embodiment of FIG. 5, both the bearing surfaces 42 and 46, which together form the bearing surface pair 50, are provided with recesses 72, as well as the bearing surfaces 44 and 48, which together define the bearing surface pair 52. In the embodiment of FIG. 5, the cooperating bearing surfaces 44 and 48 are each configured without recesses 72. In the embodiment of FIG. 19, recesses are formed only in the bearing surfaces 42 and 48. The bearing surfaces 44 and 46 are configured without recesses 72, thus recess-free. Thus, in the embodiment of FIG. 19, each bearing surface pair 50 and 52 has only one respective bearing surface 42 and 48 that is provided with at least one recess 72.

The embodiments of FIGS. 5, 7 and 19 have in common that at least one of the bearing surface pairs 50 and 52 has at least one bearing surface 42 and 46 or 42, 46, 44, and 48 or 42 and 48 that is provided with at least one recess 72.

In the embodiments of FIGS. 5, 7 and 19, the second box lock web 36 of the first connecting portion 26 is formed by a so-called box lock plate 114. It is inserted into two set-back portions 116 on the first connecting portion 26 and is welded thereto. It is inserted and welded after the respective clip 10 is formed from a wire-shaped blank by means of corresponding reshaping.

FIGS. 6 and 8 show schematically variants of different clips 10 with double box locks 64. In the embodiment of FIG. 6, the bearing surfaces 46, 44, and 68 are configured without recesses 72, thus recess-free. In contrast, recesses 72 are formed in the bearing surfaces 42, 48, and 70. Thus, at least one of the two bearing surfaces of each bearing surface pair 50, 52, and 54 is provided with at least one recess 72.

In the schematically illustrated embodiment in FIG. 8, all six bearing surfaces 42, 44, 46, 48, 68, and 70 are provided with at least one recess 72. Thus, in this embodiment too, the cooperating bearing surfaces 42, 44, 46, 48, 68, and 70 forming the respective bearing surface pairs 50, 52, and 54 are each provided with at least one recess 72.

In the embodiments of FIGS. 2, 6 and 8, the first box lock web 36 and the second box lock web 40 are configured in the form of box lock plates 114. In a manner not depicted in more detail, analogously as explained above in connection with FIGS. 5, 7, and 19 in the case of a single box lock 62, said box lock plates are inserted into and welded to set-back portions provided therefor on the two connecting portions 26 and 28.

The embodiments of medical clip 10 described above are for explanation only and are therefore not exhaustive. In particular, the number, shape, and size of the recesses 72 that are depicted and described can be combined with one another and varied as desired. For example, a groove 76 can be formed on one bearing surface and one or two chamfers 82, 84 or one or more hollow sphere portions 100 can be formed on the bearing surface that cooperates therewith. Grooves 76 parallel to a longitudinal direction 78, 80 on the one hand and transverse thereto on the other hand can be combined with one another to form a bearing surface pair 50, 52, 54.

In particular, in the schematically illustrated section views in FIGS. 5 to 8 and 19, the schematically drawn recesses 72 serve only as placeholders. Here again, grooves, chamfers, and/or hollow sphere portions as well as other forms may be provided to create the recesses 72.

In the described embodiments of medical clips 10, in particular, the desired friction reduction is achieved in that they have a ratio of the contact overlap area 74 and the projection overlap area 90 in a range of about 1/25 to about 1/3, preferably in a range of about 1/25 to about 1/4. Depending on the choice and design of the recesses 72, they are selected in shape and size such that the respective bearing surfaces that cooperate with one another are so large that the specified ratio range is fulfilled.

By reducing the contact overlap area 74 relative to the projection overlap area 98 by providing at least one recess 72 in at least one bearing surface 42, 44, 46, 48, 68, and 70 of a bearing surface pair 50, 52, or 54 of the clip 10, a transition from static friction to sliding friction can be reduced in at least one bearing surface pair 50, 52, and 54. As explained at the outset, a repeat accuracy in determining the closing force of the clip 10 is thereby increased or a repeat inaccuracy is reduced in the desired manner.

### REFERENCE NUMERAL LIST

10 clip
12 biasing element
14 first clamping arm
16 second clamping arm
18 first end
20 second end
22 first clamping arm end
24 second clamping arm end
26 first connecting portion
28 second connecting portion
30 box lock
32 first box lock perforation
34 first box lock web
36 first box lock web
38 second box lock web
40 second box lock web
42 female bearing surface
44 female bearing surface
46 male bearing surface
48 male bearing surface
50 bearing surface pair
52 bearing surface pair
54 bearing surface pair
56 bearing plane
58 bearing plane
60 bearing plane
62 single box lock
64 double box lock
66 second box lock perforation
68 female bearing surface
70 male bearing surface
72 recess
74 contact overlap area
76 groove
78 longitudinal direction
80 longitudinal direction
82 chamfer
84 chamfer
86 inclined surface
88 inclined surface
90 chamfer angle
92 width
94 first box lock web projection area
96 second box lock web projection area
98 projection overlap area
100 hollow sphere portion
102 clamping surface
104 clamping surface
106 free end
108 free end
110 coil spring
112 aneurysm clip
114 box lock plate

1. A medical clip comprising:
a first clamping arm;
a second clamping arm;
a biasing element having a first end and a second end; and
a box lock,
the first clamping arm comprising a first clamping arm end connected to the first end of the biasing element by way of a first connecting portion,
the second clamping arm comprising a second clamping arm end connected to the second end of the biasing element by way of a second connecting portion,
the box lock comprising the first connecting portion and the second connecting portion,
the box lock having at least one first box lock perforation arranged or formed on the first connecting portion and delimited by two first box lock webs,
the box lock also having at least one second box lock web comprised by the second connecting portion and passing through the at least one first box lock perforation,
the first box lock perforation comprising two female bearing surfaces facing toward one another,
the at least one second box lock web comprising two male bearing surfaces facing away from one another and toward the two female bearing surfaces,
the two male bearing surfaces and the two female bearing surfaces forming at least two bearing surface pairs,
each bearing surface pair comprising one of the two male bearing surfaces and one of the two female bearing surfaces,
the male bearing surface and the female bearing surface of each bearing surface pair extending in parallel with one another and defining a bearing plane,
the two first box lock webs comprising a first perpendicular projection that projects into the bearing plane, the first perpendicular projection defining a first box lock web projection area,
the at least one second box lock web comprising a second perpendicular projection that projects into the bearing plane, the second perpendicular projection defining a second box lock web projection area,
the first box lock web projection area and the second box lock web projection area overlapping and defining a projection overlap area,
at least one of the two cooperating bearing surfaces of at least one of the at least two bearing surface pairs has at least one recess such that an actual size of the bearing surface in parallel with the bearing plane, said bearing surface being defined by the box lock web comprising the at least one recess, is smaller than the box lock web projection area projected from the box lock web comprising the at least one recess into the bearing plane,
the two cooperating bearing surfaces of at least one bearing surface pair being able to abut against one another or are abutting in surface-to-surface contact against one another to define a contact overlap area, and
a ratio of the contact overlap area and the projection overlap area being 1/25 to 1/3.

2. The medical clip according to claim 1, wherein the at least one recess is a groove in the bearing surface or a chamfer on the bearing surface.

3. The medical clip according to claim 2, wherein the groove or the chamfer extends parallel to a longitudinal direction defined by the respective box lock web.

4. The medical clip according to claim 2, wherein the chamfer defines an inclined surface, which together with the bearing plane defines a chamfer angle that is obtuse.

5. The medical clip according to claim 4, wherein the chamfer angle is between 140° and 175°.

6. The medical clip according to claim 2, wherein the groove extends transversely to a longitudinal direction defined by the respective box lock web.

7. The medical clip according to claim 1, wherein the at least one recess is a hollow sphere portion.

8. The medical clip according to claim 1, wherein the at least one recess is formed by a box lock web perforation passing through the box lock web.

9. The medical clip according to claim 8, wherein, the box lock web perforation has a circular cross section.

10. The medical clip according to claim 1, wherein at least one of the two cooperating bearing surfaces of at least one of the at least two bearing surface pairs has a plurality of recesses.

11. The medical clip according to claim 10, wherein the plurality of recesses are of identical configuration.

12. The medical clip according to claim 10, wherein at least a portion of the plurality of recesses differ in their shape and/or size.

13. The medical clip according to claim 1, wherein both cooperating bearing surfaces of each bearing surface pair comprise at least one recess.

14. The medical clip according to claim 1, wherein a width of the box lock webs, without taking into account the at least one recess, transverse to a longitudinal direction defined by the respective box lock web, is in a range of 0.9 mm to 2.1 mm.

15. The medical clip according to claim 1, wherein the box lock is a single box lock and the second connecting portion comprises only one second box lock web.

16. The medical clip according to claim 1, wherein:
the box lock is a double box lock and the second connecting portion comprises two second box lock webs that laterally delimit a second box lock perforation, and
one of the two first box lock webs passes through the second box lock perforation, and one of the two second box lock webs passes through the first box lock perforation.

17. The medical clip according to claim 1, wherein the first clamping arm, commencing from the first clamping arm end, and the second clamping arm, commencing from the second clamping arm end, are of rectilinear or curved or angled configuration in a direction toward free ends thereof.

18. The medical clip according to claim 1, wherein at least one of:
a) the clip is made of a metallic material; and
b) the cooperating bearing surfaces are made of a metallic material.

19. The medical clip according to claim 1, wherein the first and/or second box lock webs have a rectangular cross section, without taking into account the at least one recess.

20. The medical clip according to claim 1, wherein at least one bearing surface of the two cooperating bearing surfaces of one bearing surface pair is provided with a coating.
